(19) United States
(12) Patent Application Publication
(10) Pub. No.: US 2021/0038466 A1
(43) Pub. Date: Feb. 11, 2021

(54) CUPPING DEVICE FOR ELECTRICAL MYOFASCIAL DECOMPRESSION

(71) Applicant: OLYMPUS INNOVATIONS, LLC, Murray, UT (US)

(72) Inventors: Daniel G. Stewart, Jr., Murray, UT (US); Jordan W. Meier, Murray, UT (US); James Beagley, Murray, UT (US)

(21) Appl. No.: 16/987,989

(22) Filed: Aug. 7, 2020

Related U.S. Application Data
(60) Provisional application No. 62/884,887, filed on Aug. 9, 2019.

(51) Int. Cl. A61H 9/00; A61N 1/36

(52) U.S. Cl. CPC A61H 9/0057; A61H 2201/1409; A61H 2201/10; A61N 1/36014

(57) ABSTRACT

A therapy device is configured to provide myofascial decompression and transcutaneous electrical nerve stimulation simultaneously. The device includes a cup structure capable of holding a negative pressure within an interior chamber. A plurality of conductive contacts are coupled to the cup structure. The conductive contacts are disposed so as to be at least partially exposed on a bottom surface of the cup structure.

### CROSS-REFERENCE TO RELATED APPLICATIONS

This application claims priority to and the benefit of U.S. Provisional Patent Application No. 62/884,887, filed Aug. 9, 2019 and titled “Cupping Device for Electrical Myofascial Decompression,” the entirety of which is incorporated herein by this reference.

### BACKGROUND

Myofascial decompression (MFD), also at times referred to as “cupping,” is a technique for addressing dysfunctions of muscle, fascia, and other connective tissues through the selective application of negative pressure. During MFD therapy, a cup is placed open-side-down on a subject's skin. Air is then evacuated from the cup, usually through a valve on the closed end of the cup. The resulting negative pressure lifts the skin and underlying tissues and thereby allows for release of the fascia and muscle tissues. MFD may be used, for example, to help “break up” scar tissue and promote healing in the fascia.

Dysfunctions of the fascia or associated connective tissues often cause a decrease in range of motion and function, which can lead to an increase in pain and discomfort. MFD is intended to reduce fascial plane restrictions and connective tissue matrix dysfunctions by lifting adhesions between tissue layers. This can allow for greater blood flow and metabolic exchange at the targeted tissues, which in turn can reduce scar tissue formation, release trigger points, decrease tightness, decrease pain, and/or increase rate of healing.

Transcutaneous electrical nerve stimulation (TENS) is a therapy that involves the application of relatively low electric currents to targeted treatment areas, usually with the intent to treat pain and/or relax tense muscles. TENS therapy involves placement of electrodes on the skin at the target site to allow delivery of current to the tissues. The applied current can thereby stimulate underlying nerves and/or cause mild muscle contractions.

While both MFD and TENS have their benefits, several limitations remain. For example, some patients may find one or both of these therapies to be uncomfortable. TENS therapy requires the use of electrode pads, which can be difficult to place, difficult or impossible to clean, and typically must be replaced relatively frequently. Further, many patients may simply fail to see desired results with these therapies as they are conventionally practiced.

Accordingly, there is an ongoing need for improved therapeutic devices and methods which can be utilized to effectively treat targeted tissues and thereby provide increased range of motion, decreased pain, increased tissue healing, and/or reduced scar tissue formation.

### SUMMARY

The present disclosure describes devices and methods for providing both MFD and TENS therapies simultaneously. The embodiments described herein are beneficially capable of providing synergistic effects that are greater than with conventional MFD or TENS alone, or even greater than with sequential administration of MFD and TENS therapies. Embodiments described herein may promote increased range of motion, decreased pain, increased tissue healing, and/or reduced formation of scar tissue, for example.

In one embodiment, a therapy device includes a cup structure having an open end and a closable end. The cup structure is preferably rigid and capable of holding a negative pressure within an interior chamber when a bottom surface of the open end is positioned on a patient's skin. A plurality of conductive contacts are coupled to the cup structure. Each conductive contact is disposed so as to be at least partially exposed on the bottom surface of the cup structure to allow current to pass to the bottom surface and then to the patient during use.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIGS. 1A and 1B illustrate an exemplary therapy device capable of providing MFD and TENS therapies simultaneously;

FIG. 1C illustrates a detailed view of a receptacle and conductive contact of the therapy device;

FIG. 2 illustrates an alternative therapy device;

FIG. 3 illustrates a conductive ring structure that may be utilized as part of a therapy device; and

FIGS. 4A and 4B illustrate another embodiment of a therapy device.

### DETAILED DESCRIPTION

### Overview of Exemplary Cupping Devices

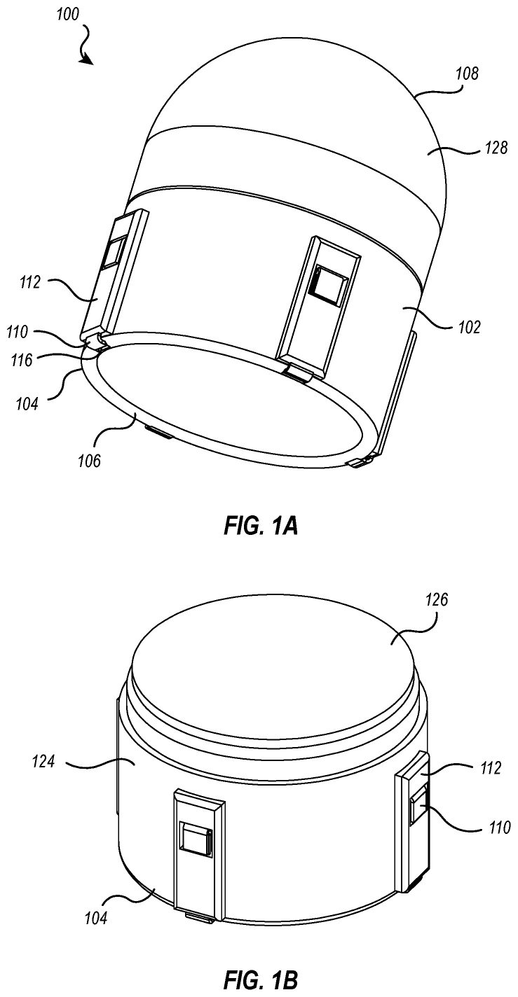

FIGS. 1A through 1C illustrate one example of a therapy device 100 configured to provide simultaneous MFD and TENS treatments. The illustrated device 100 includes a cup structure 102 having an open end 104 and a closable end 108. The open end 104 has a bottom surface 106 that interfaces with a patient's skin when the device is placed on the patient during use. The closable end 108 may include a valve (not shown) which allows air to be evacuated from the interior chamber of the cup structure 102. Such a valve may additionally or alternatively be located at other portions of the cup structure 102.

The cup structure 102 may be formed from any clinically suitable material. The material should preferably provide sufficient rigidity such that the cup structure 102 maintains its shape when a negative pressure is applied during use of the device 100, though other embodiments may utilize non-rigid materials when suitable for particular applications. The cup structure 102 is preferably also at least partially transparent so as to enable clinicians to visualize the underlying skin and amount of lift during use of the device 100. Typically, the cup structure 102 is formed of a rigid polymer material, such as rigid polymers including polycarbonate, polystyrene, polypropylene, polyethylene, polyvinyl chloride, or other polymers that can be made sufficiently rigid. Other embodiments may at least partially include other materials such as metals or ceramics (including glass).

The cup structure 102 is shown here as having a circular cross section and cylindrical shape, with a curved, generally dome-shaped closable end 108. Although such a shape is generally convenient for the therapy device 100, other embodiments may take other shapes. For example, some embodiments may have an ovoid or polygonal cross section, and/or may have a closable end 108 with one or more protrusions, angled surfaces, or other elements different from a rounded, dome shape.

The therapy device 100 also includes a plurality of conductive contacts 110 integrally coupled to the cup structure 102. The conductive contacts 110 are arranged so that at least a portion is disposed along the bottom surface 106 where direct contact will be made with the patient during use of the device 100.

The conductive contacts 110 may be formed from any conductive material suitable for clinical use. Examples include aluminum, gold, silver, copper, platinum, other conductive metals, mixtures thereof, and alloys thereof. Conductive polymer materials (e.g., doped polymers) may also be utilized.

In preferred embodiments, the conductive contacts 110 are formed from the same material. That is, the device 100 is not intended for or designed to provide current based on galvanic differences between adjacent, dissimilar metals. Similarly, the presently described embodiments preferably space each of the conductive contacts from one another and generally seek to avoid configurations that would lead to galvanic corrosion.

Providing electrical current through the use of dissimilar metals was not found to generate clinically useful levels of current. Rather, the presently described embodiments utilize conductive contacts 110 that are arranged to allow for attachment to the leads of a TENS unit. A conventional TENS unit is configured to provide current in the mA range (e.g., 1 to 80 mA). This is significantly greater than the barely detectable galvanic microcurrents resulting from closely spaced, dissimilar metals alone.

The conductive contacts 110 are disposed within receptacles 112 arranged around the outer surface of the cup structure 102. The receptacles 112 and corresponding conductive contacts 110 may be arranged in a radially symmetric fashion, as shown. Alternatively, the receptacles 112 and corresponding conductive contacts 110 may be arranged non-symmetrically according to particular treatment application needs.

The receptacles 112 function to receive and hold the corresponding conductive contacts 110 at desired locations upon the cup structure 102. As explained in greater detail below, the receptacles 112 include a bottom opening 114 and an upper opening 118 through which the conductive contacts 110 may be routed and/or accessed. In the illustrated embodiments, the receptacles 112 extend in a direction parallel to a longitudinal axis of the device (i.e., an axis extending from the open end 104 to the closable end 108).

This arrangement of the receptacles 112 allows them to position the corresponding conductive contacts 110 with a lower section that can be coincident with the bottom surface 106 and an upper section located further upwards and out of the way. In this manner, electrical connection(s) between the conductive contacts 110 and TENS unit leads can be made without interfering with the lower sections of the cup structure 102 in contact with the patient.

The cup structure 102 may be formed from a single, integral piece. Alternatively, multiple sections may be separately formed and assembled together to form the cup structure 102. In the illustrated embodiment, a lid section 128 may be separated from a base section 124. FIG. 1B illustrates the device 100 with the lid section 128 removed. A diaphragm 126 may be included between the base section 124 and lid section 128 and utilized to transfer a negative pressure to the interior chamber of the base section 124. Other embodiments may omit such a diaphragm and instead utilize one-way valves and/or other pneumatic elements to apply negative pressure.

FIG. 1C is a detailed view of a receptacle 112 and conductive contact 110 of the device 100. As shown, the conductive contact 110 extends out of the bottom opening 114 to be coincident with the bottom surface 106 of the cup structure 102. In this way, a bottom portion of the conductive contact 110 is available to contact the patient's skin when the bottom surface 106 of the cup structure 102 is positioned on the patient's skin.

The conductive contact 110 also extends upward through the receptacle 112 to the upper opening 118, where an upper section of the conductive contact 110 is exposed. During use, leads of a TENS device may be electrically coupled to the exposed upper sections of the conductive contacts, allowing electric current to pass to the lower sections of the conductive contacts that are coincident with the bottom surface 106.

The bottom surface 106 of the cup structure 102 may also include slots 116 with a size and shape that allow the corresponding lower sections of the conductive contacts 110 to be positioned therein. The slots 116 allow the bottom surface 106 to be substantially planar even with the inclusion of the lower sections of the conductive contacts 110.

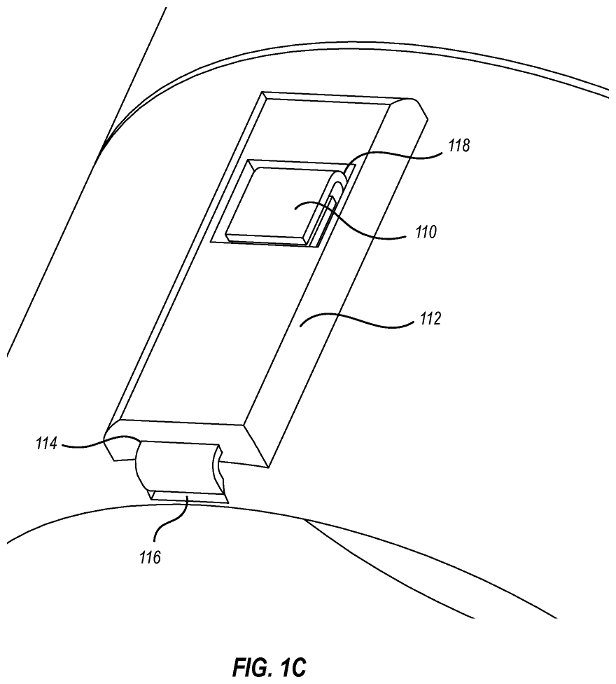

FIG. 2 illustrates another embodiment of a therapy device 200. The therapy device 200 may be similar to the therapy device 100 and may include any of the features described above. As with the previous embodiment, the device 200 includes a cup structure 202 with an open end 204, a bottom surface 206, and a closable end 208. The cup structure 202 also includes a nozzle portion 220 disposed at the closable end 208. The nozzle portion 220 may hold a valve (not shown) and/or be configured to couple to a pump or other suitable pneumatic fixtures known in the art to allow the generation of negative pressure within the interior chamber of the cup structure 202.

FIG. 2 also illustrates a different configuration of receptacles 212. In the embodiment of FIG. 2, the upper openings 218 face substantially upward (rather than substantially outward as with the upper openings 118 of the FIG. 1 device). The particular orientation of the upper openings can beneficially be tailored to particular application needs and/or user preferences. For example, certain types of TENS lead connections (e.g., alligator clips, pins, snap connectors, plugs, banana connection) may be more readily connected to the conductive contacts with an upward facing upper opening as opposed to an outward facing upper opening, or vice versa.

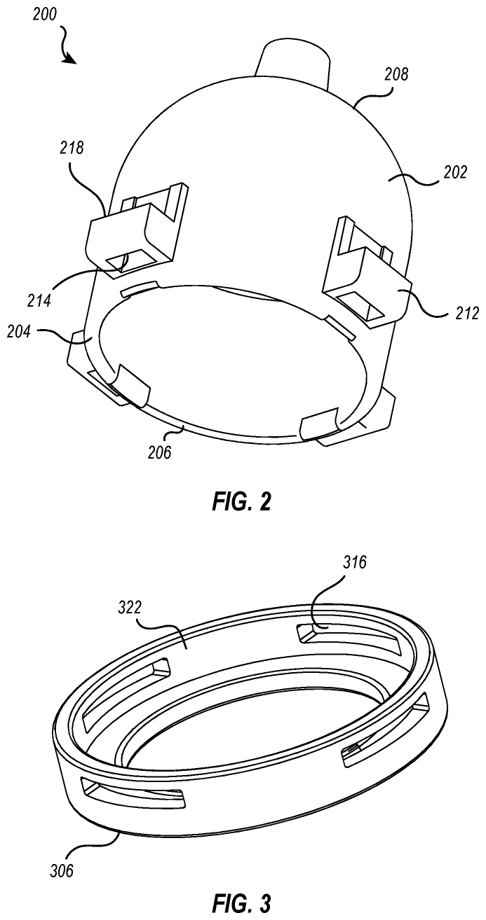

FIG. 3 illustrates a component that may be utilized in another embodiment of a therapy device. The illustrated component is a conductive ring 322 that may be coupled to a cup structure to form the bottom section and bottom surface 306 of the device. Because ring 322 is itself conductive, the bottom surface 306 may be uniformly formed of the same material (i.e., omitting slots, indentations, and such).

Slots 316 may be formed in an outer surface of the ring 322. The slots 316 can directly receive a TENS lead, or alternatively can receive a portion of a conductive contact as in embodiments described above. Such conductive contacts may then be routed through a receptacle or otherwise coupled to or integrated with the cup structure, such receptacles having an upper opening for providing access to an upper section of the conductive contact as described above.

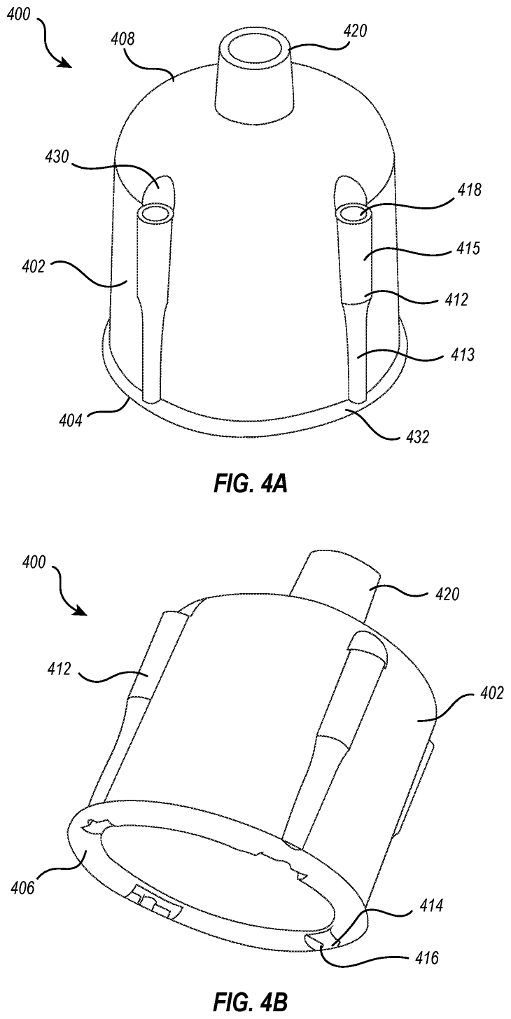

FIGS. 4A and 4B illustrate another embodiment of a therapy device 400 that may be utilized for simultaneous MFD and TENS treatments. The therapy device 400 may be similar to the other therapy devices described herein, and except where noted otherwise, may incorporate any of the features described above.

As with the other embodiments described herein, the device 400 includes a cup structure 402 with an open end 404 and a closable end 408. The closable end 408 includes a nozzle portion 420 in which a valve (not shown) may be located and/or at which various pneumatic fixtures may be attached for generating a negative pressure within the device. The open end 406 forms a bottom surface 406 which directly contacts the patient's skin during use of the device 400. In this embodiment, the open end 404 may also be at least partially defined by a lip 432 that provides an outwardly protruding edge along the open end 404.

In the illustrated embodiment, the receptacles 412 are partially formed within the sidewall of the cup structure 402 such that interior spaces of the receptacles extend at least partially within the sidewall of the cup structure 402. This design was found to beneficially integrate the receptacles 412 with the cup structure 402, make efficient use of space, and leave enough outward protrusion from the receptacles 412 to provide tactile surfaces for gripping and manipulating the device 400.

The receptacles 412 have upper openings 418 that face substantially upward. The cylindrical shape of the receptacles 412 is also beneficial for receiving TENS lead connectors, and in particular for receiving common cylindrical pin connectors (e.g., typically 2 mm or 3 mm in size). By positioning the upper openings 418 in this manner, it has been found that coupling standard TENS leads to the device is straightforward and allows the lead connections and wires to be positioned in a manageable and desirable position.

For example, when connected to the device 400, the lead contacts will extend substantially upward out of the upper openings 418, and the wires will remain relatively near one another for easy management. In contrast, having a number of clips, connectors, and/or wires radiating outward from the device in multiple directions can lead to more cumbersome use of the device.

The receptacles 412 can thus function to receive and hold the pins within upper sections 415 of the receptacle, while the corresponding conductive contact is integrated within a lower section 413 of the receptacle. As shown, the upper section 415 may have a larger diameter than the lower section 413 so as to limit the downward motion of an inserted pin.

In this embodiment, the upper end of the conductive contact need not be exposed because it may sit at a height that is lower than the upper opening 418 of the receptacle 412. For example, the upper openings 418 may be positioned at a height that is greater than about 50% of the height between the open end 404 and the closable end 408 (not including the nozzle portion 420), or more preferably greater than about 60% or greater than about 75% of the height between the open end 404 and the closable end 408.

The cup structure 402 may include indentations 430 disposed adjacent the upper openings 418. The indentations 430 extend inward and may function to provide clearance around the upper openings 418 for easier insertion of TENS leads. The illustrated embodiment also orients the receptacles in a direction that is substantially parallel with the longitudinal axis of the device.

As best shown in FIG. 4B, the bottom opening 414 of the receptacle 412 can extend all the way to the bottom surface 406. The bottom opening 414 may be coincident with a corresponding slot 416 so that an integrated conductive contact (not shown in this view) can extend through the bottom opening 414 and into the slot 416 and thus be disposed for contact with the patient along with the rest of the bottom surface 406.

Although the examples shown and described herein are configured to receive four lead connectors, other embodiments may be configured for a different number of lead connections. For example, some embodiments may include two receptacles and two corresponding conductive contacts. Other embodiments may include more than four receptacles and corresponding conductive contacts.

In addition, some embodiments may be configured for a number of lead connections, but in use need not necessarily utilize all of them. For example, an embodiment as illustrated that includes four receptacles may be used in some applications with only two lead connections made to only two of the receptacles.

In some embodiments, the receptacles may include a length of a flexible, insulated conductor. For example, the receptacles may extend away from the cup structure by way of a flexible, insulated conductor. Such an embodiment may provide additional distance between the cup structure and the electrical contact points at which TENS leads can be contacted.

### Methods of Use

During use, a therapy device such as described herein may be positioned on a patient such that the bottom surface is in contact with the patient's skin at or near a targeted area for treatment. The device is also coupled to a current source such that current is passable to the conductive contacts of the device. Once positioned, a pump or other pneumatic device may be utilized to evacuate air from the interior chamber to create negative pressure. This lifts the skin and underlying fascia (e.g., about 10 cm depending on particular patient needs, pain tolerances, and treatment goals).

Current is then passed to the conductive contacts of the device, and thus from the conductive contacts to the patient. In some implementations, a conductive (and also optionally adhesive) pad is positioned on the patient. The pad may be utilized to form an electrical pathway external to the perimeter/bottom surface of the cup structure and the patient. In some implementations, the conductive pad may form an electrical pathway between the bottom surface of the cup structure and the patient.

One or more therapy devices as described herein may be moved along targeted areas of the patient during the treatment. Additionally, or alternatively, one or more therapy devices may be applied statically and left in place at a targeted position for a determined treatment period. A typical treatment may last about 8 to 10 minutes.

During treatment, one or more different TENS profiles may be applied. The intensity (amount of current), pulse frequency, and/or pulse width may be varied to provide desired treatments. Different treatment protocols and TENS contact point arrangements may be utilized such as premodulated bipolar, interferential quadpolar, and/or other arrangements known in the art. In some implementations, two or more therapy devices could be electrically connected and used in conjunction with each other (e.g., as though they are each a separate TENS pad) to provide a desired treatment protocol.

### CONCLUSION

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, or less than 1% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may include properties, features (e.g., ingredients, components, members, elements, parts, and/or portions) described in other embodiments described herein. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. A therapy device configured to provide myofascial decompression and transcutaneous electrical nerve stimulation simultaneously, the device comprising:
a cup structure having an open end and a closable end and being configured to hold a negative pressure within an interior chamber when positioned on a patient's skin, the open end having a bottom surface configured for interfacing with the patient's skin; and
a plurality of conductive contacts coupled to the cup structure, each conductive contact disposed so as to be at least partially exposed on the bottom surface of the cup structure.

2. The device of claim 1, wherein the closable end of the cup structure includes a valve.

3. The device of claim 2, wherein the closable end of the cup structure includes a nozzle section of reduced diameter, and wherein the valve is disposed within the nozzle section.

4. The device of claim 1, wherein the cup structure is rigid so as to maintain shape when a negative pressure is applied within the cup structure.

5. The device of claim 1, wherein the cup structure comprises a lid section formed separately from and being separable from a body section, the lid section including the closable end and the body section including the open end.

6. The device of claim 1, further comprising a plurality of receptacles arranged upon the cup structure, each receptacle being configured to receive and position a corresponding conductive contact.

7. The device of claim 6, wherein each receptacle is arranged around an outer surface of the cup structure in a radially symmetric fashion.

8. The device of claim 6, wherein the receptacles extend in a direction parallel to a longitudinal axis of the cup structure.

9. The device of claim 6, wherein the receptacles each extend from a bottom opening to an upper opening.

10. The device of claim 9, wherein the bottom openings are coincident with the bottom surface of the cup structure.

11. The device of claim 9, wherein for each receptacle, a radially inward indentation is formed in the cup structure near the upper opening of the receptacle.

12. The device of claim 9, wherein the receptacles are formed with a cylindrical cross section so as to receive lead wire pins.

13. The device of claim 12, wherein each receptacle defines an interior space that extends at least partially into a sidewall of the cup structure.

14. The device of claim 9, wherein each conductive contact is fully disposed within its corresponding receptacle except where exposed at the bottom surface of the cup structure.

15. The device of claim 1, wherein the bottom surface comprises slots each configured to receive a portion of a corresponding conductive contact, the slots being sized such that the bottom surface of the cup structure is substantially flush with the received portions of the conductive contacts when each conductive contact is positioned within its corresponding slot.

16. The device of claim 1, wherein each conductive contact is formed from the same material.

17. The device of claim 1, wherein at least a portion of the conductive contacts are exposed along an outer surface of the cup structure.

18. A method of simultaneously applying myofascial decompression and transcutaneous electrical nerve stimulation to a patient, the method comprising:
providing a negative pressure device capable of providing a negative pressure to targeted tissue areas;
coupling the negative pressure device to a current source such that current is passable to the conductive contacts of the device;
positioning the device on a patient's skin;
evacuating air from the negative pressure device to create negative pressure; and
passing current to the conductive contacts of the negative pressure device while a negative pressure is maintained within the interior chamber.

19. The method of claim 18, further comprising moving the negative pressure device along the patient's skin while maintaining a negative pressure.

20. The method of claim 18, further comprising applying a conductive gel to a targeted portion of the patient's skin prior to positioning the negative pressure device thereon, applying a conductive pad to the patient at a position external to the bottom surface of the cup structure, or both.
